(19) United States
(12) Patent Application Publication
(10) Pub. No.: US 2023/0001194 A1
(43) Pub. Date: Jan. 5, 2023

(54) METHODS AND COMPOSITIONS FOR INCREASING CIRCULATION DURING TREATMENT WITH ALTERNATING ELECTRIC FIELDS

(71) Applicant: Novocure GMBH, Root D4 (CH)

(72) Inventor: Ori Farber, Haifa (IL)

(21) Appl. No.: 17/841,987

(22) Filed: Jun. 16, 2022

Related U.S. Application Data
(60) Provisional application No. 63/216,927, filed on Jun. 30, 2021.

(51) Int. Cl. A61N 1/36; A61K 45/06

(52) U.S. Cl. CPC A61N 1/36002; A61K 45/06

(57) ABSTRACT

Disclosed are methods of increasing blood circulation at a target site of a subject comprising exposing a target site of the subject to an alternating electric field for a period of time, the alternating electric field having a frequency and field strength, and exposing the target site of the subject to a vasodilator, wherein the vasodilator increases blood circulation at the target site of the subject. Disclosed are methods of maintaining or decreasing temperature at a target site of a subject comprising exposing a target site of the subject to an alternating electric field for a period of time, the alternating electric field having a frequency and field strength, and exposing the target site of the subject to a vasodilator, wherein the vasodilator increases circulation at the target site of the subject, wherein increased circulation maintains or decreases temperature at the target site.

### BACKGROUND

The use of alternating electric fields as a cancer treatment are well known. Due to the electric currents in the alternating electric fields, heat can be expected to be generated. Thus, proper cancer treatments can depend on finding the appropriate electric current to be used for the alternating electric fields to both provide effective treatment and not provide harmful effects from the electric currents. One such harmful effect is increased heat at the site of the alternating electric fields.

The ability of the flow of large blood volumes to promote heat dissipation is well known. Thus, described herein are methods of creating a mechanism for increased heat dissipation in order to prevent too much heat generated from alternating electric fields to cause harmful effects during treatment.

### BRIEF SUMMARY

Disclosed are methods of increasing blood circulation at a target site of a subject comprising exposing a target site of the subject to an alternating electric field for a period of time, the alternating electric field having a frequency and field strength, and contacting the target site of the subject to a vasodilator, wherein the vasodilator increases blood circulation at the target site of the subject.

Disclosed are methods of maintaining or decreasing temperature at a target site of a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein increased circulation maintains or decreases temperature at the target site.

Disclosed are methods of enhancing the efficacy of an alternating electric field at a target site of a subject comprising exposing a target site of the subject to an alternating electric field for a period of time, the alternating electric field having a frequency and field strength, and contacting the target site of the subject to a vasodilator, wherein the vasodilator increases circulation at the target site of the subject.

Disclosed are methods of treating cancer in a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein the target site comprises one or more cancer cells.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a vasodilator” includes a plurality of such vasodilators, reference to “the target site” is a reference to one or more target sites and equivalents thereof known to those skilled in the art, and so forth.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g. a target site). In some aspects, the alternating electrical field can be in a single direction or multiple directional. In some aspects, alternating electric fields can be delivered through two pairs of transducer arrays that generate perpendicular fields within the treated heart. For example, for the Optune™ system (an alternating electric fields delivery system) one pair of electrodes is located to the left and right (LR) of the heart, and the other pair of electrodes is located anterior and posterior (AP) to the heart. Cycling the field between these two directions (i.e., LR and AP) ensures that a maximal range of cell orientations is targeted. In some aspects, an alternating electric field can be referred to as Tumor Treating Field (TTF).

In-vivo and in-vitro studies show that the efficacy of alternating electric fields therapy increases as the intensity of the electrical field increases. Therefore, optimizing array placement on the area of a patient's target site to increase the intensity in the desired target site can be performed (e.g. with the Optune system). Array placement optimization may be performed by “rule of thumb” (e.g., placing the arrays on the target site as close to the desired region of the target site (e.g. cancer cells) as possible), measurements describing the geometry of the patient's target site dimensions. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the head as a function of the positions of the array and, in some aspects, take account for variations in the electrical property distributions within the heads of different patients.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field.

As used herein, a “target site” is a specific site or location within or present on a subject or patient. For example, a “target site” can refer to, but is not limited to a cell, population of cells, organ, tissue, tumor, or cancer cell. In some aspects, organs include, but are not limited to, lung, brain, pancreas, abdominal organs (e.g. stomach, intestine), ovary, breast, uterus, prostate, bladder, liver, colon, or kidney. In some aspects, a cell or population of cells include, but are not limited to, lung cells, brain cells, pancreatic cells, abdominal cells, ovarian cells, liver cells, colon cells, or kidney cells. In some aspects, a “target site” can be a tumor target site.

By “treat” is meant to administer or apply a therapeutic, such as alternating electric fields, to a subject, such as a human or other mammal (for example, an animal model) in order to prevent or delay a worsening of the effects of a disease, disorder, or condition, or to partially or fully reverse the effects of the disease, disorder, or condition. In some aspects, the disease, disorder, or condition can be a cancer or circulatory condition. In some aspects, the subject is in need of treatment to increase circulation. In some aspects, the subject in need of treatment for cancer or for an increased susceptibility for developing cancer.

By “prevent” is meant to minimize the chance that a subject who has an increased susceptibility for developing a disease, disorder, or condition will develop the disease, disorder, or condition.

As used herein, the terms “administering” and “administration” refer to any method of providing a therapeutic, such as a vasodilator to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject. In some aspects, administering comprises exposing or contacting. Thus, in some aspects, exposing a cancer cell to alternating electrical fields means administering alternating electrical fields to the cancer cell.

An “effective amount” of a therapeutic (e.g. vasodilator) is that amount of therapeutic which is sufficient to provide a beneficial effect to the subject to which the therapeutic is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. An “effective amount” of a vasodilator is that amount sufficient to effectively increase the blood flow in a blood vessel.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Increasing Blood Circulation

Increasing blood circulation, or blood flow, at a target site can be beneficial. For example, blood is known to dissipate heat via normal blood flow. Thus, if a treatment that can generate heat is administered to a target site, in can be beneficial to increase blood circulation at the target site to help move some of the heat out of the target site in order to allow for more treatment. In some aspects, blood circulation and blood flow can be used interchangeably. In some aspects, as used throughout, blood circulation and circulation can be used interchangeably.

Disclosed are methods of increasing blood circulation at a target site of a subject comprising exposing a target site of the subject to an alternating electric field for a period of time, the alternating electric field having a frequency and field strength, and contacting the target site of the subject to a vasodilator, wherein the vasodilator increases blood circulation at the target site of the subject. In some aspects, disclosed are methods of increasing blood circulation at a target site of a subject comprising exposing a target site of the subject to an alternating electric field for a period of time, the alternating electric field having a frequency and field strength, and administering a vasodilator to the subject, wherein the vasodilator increases blood circulation at the target site of the subject. Such administration can be systemic or local (e.g. transdermal).

In some aspects, the target site comprises a cancer cell. In some aspects, the cancer cell is a glioblastoma cell or lung metastatic carcinoma cell. In some aspects, the cancer cell can be any cancer cell.

In some aspects, the target site is exposed to the alternating electric field and vasodilator simultaneously. In some aspects, the target site is exposed to the alternating electric field and vasodilator consecutively. For example, in some aspect, the target site is exposed to the alternating electric field prior to or after the target site is exposed to the vasodilator. In some aspects, consecutive exposure of the target site to both the alternating electric field and vasodilator can mean the exposure to each occurs within 1, 5, 10, 15, 20, 25, 30, 35, 40, 45, 50, 55, or 60 minutes; 1, 2, 3, 4, 5, 6, 7, 8, 9, 10, 11, 12, 13, 14, 15, 16, 17, 18, 19, 20, 21, 22, 23, or 24 hours; or 1, 2, 3, 4, 5, 6, 7, 8, 9, 10, 11, 12, 13, or 14 days of each other.

In some aspects, the vasodilator can be any agent (e.g. compound, peptide, nucleic acid) that opens, or dilates, blood vessels. The dilation of blood vessels can allow blood to flow more easily through the blood vessels. Thus, in some aspects, the vasodilator can be administered in a therapeutically effective amount to increase the flow of blood through blood vessels. In some aspects, the vasodilator can be administered systemically or locally (e.g. transdermal). In some aspects, the vasodilator is nitroprusside, nitroglycerin, minoxidil, or hydralazine. Examples of additional vasodilators that can be use include, but are not limited to, amrinone, arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, and sodium nitroprusside. Centrally acting agents include clonidine, quanaberz, and methyl dopa. Alpha adrenoceptor blocking agents include indoramin, phenoxybenzamine, phentolamine, and prazosin. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion blocking agents include pentolinium and trimetaphan. Calcium channel blockers include amlodipine, diltiazem, felodipine, isradipine, nicardipine, nifedipine, nimodipine, and verapamil. Prostaglandins including: prostacyclin, thrombuxane A2, leukotrienes, PGA, PGA1, PGA2, PGE1, PGE2, PGD, PGG, and PGH can also be used. Angiotensin II analogs can also be used and include saralasin. Other suitable vasodilators that can be used include nitroglycerin, labetalol, thrazide, isosorbide dinitrate, pentaerythritol tetranitrate, digitalis, hydralazine, diazoxide, and sodium nitroprusside, derivatives of these and combinations of the foregoing. In some aspects, the disclosed vasodilators can be a single vasodilator or the combination of two or more vasodilators.

In some aspects, the vasodilator can be administered using any of the routes of administration described throughout. In some aspects, the vasodilator can be administered intravenously or transdermally. In some aspect, contacting the target site of the subject to a vasodilator comprises transdermal administration of the vasodilator to the subject at the target site. In some aspects, contacting a target site of a subject to a vasodilator can occur by administering the vasodilator at a location other than the target site so long as the route of administration allows for vasodilation at the target site. For example, in some aspects, the vasodilator can be present in a gel, or similar form, used when applying the alternating electric field to a target site.

In some aspects, the vasodilator can be a negative pressure environment that vasodilates the vasculature. For example, using a negative pressure device to create vasodilation can be used in the extremities of a subject.

In some aspects, the increase in blood circulation is 1% greater than circulation prior to exposure to the vasodilator. In some aspect, an increase in blood circulation is 1%, 5% or 10% greater than circulation prior to exposure to the vasodilator. The increase in circulation can be measured relative to circulation prior to treatment with a vasodilator or relative to circulation after treatment with a control formulation lacking a vasodilator. In some aspect, an increase in blood circulation can be measured using laser Doppler velocimetry, which typically outputs a voltage that is proportional to the velocity of cells moving through the blood.

In some aspects, the target site is the subject's head, chest, or extremity.

In some aspects, the temperature at the target site after exposing the target site to an alternating electric field is not increased compared to the temperature at the target site prior to exposure to the alternating electric field. In some aspects, no increase in temperature can include a temperature that remains within less than one degree difference from the temperature prior to the alternating electric field. Thus, in some aspects, the temperature at a target site after exposing the target site to an alternating electric field is maintained. In some aspects, the temperature at the target site after exposing the target site to an alternating electric field is decreased compared to the temperature at the target site prior to exposure to the alternating electric field. In some aspects, a decrease in temperature can include a one degree drop in temperature or more.

In some aspects, the period of time, frequency or field strength of the alternating electrical field can be increased from a first exposure period of time, frequency or field strength of the alternating electrical field. For example, the vasodilators can maintain or decrease the temperature at the target site well enough to allow for an extended period of time for exposure to the alternating electrical field and/or to allow for increased frequency and/or field strength of the alternating electrical field over the exposure time period. The heat that could be caused by prolonged exposure to the alternating electrical field or exposure to the alternating electrical field at a frequency or field strength higher than normal can be dissipated by the vasodilator. Thus, in some aspects, treatment with alternating electrical fields can be altered when combined with a vasodilator.

### C. Methods of Maintaining or Decreasing Temperature

Maintaining normal body temperature at a target site or decreasing the temperature at the target site during certain situations that can cause elevated temperatures can be beneficial. For example, maintaining or decreasing temperatures during treatments that can increase temperatures can allow for prolonged treatment periods or

Disclosed are methods of maintaining or decreasing temperature at a target site of a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein increased circulation maintains or decreases temperature at the target site. In some aspects, disclosed are methods of maintaining or decreasing temperature at a target site of a subject comprising exposing a target site of the subject to an alternating electric field for a period of time, the alternating electric field having a frequency and field strength, and administering a vasodilator to the subject, wherein the vasodilator increases circulation at the target site of the subject, wherein increased circulation maintains or decreases temperature at the target site

In some aspects, the target site comprises a cancer cell. In some aspects, the cancer cell is a glioblastoma cell or lung metastatic carcinoma cell. In some aspects, the cancer cell can be any cancer cell.

In some aspects, the target site is exposed to the alternating electric field and vasodilator simultaneously. In some aspects, the target site is exposed to the alternating electric field and vasodilator consecutively. For example, in some aspect, the target site is exposed to the alternating electric field prior to or after the target site is exposed to the vasodilator. In some aspects, consecutive exposure of the target site to both the alternating electric field and vasodilator can mean the exposure to each occurs within 1, 5, 10, 15, 20, 25, 30, 35, 40, 45, 50, 55, or 60 minutes; 1, 2, 3, 4, 5, 6, 7, 8, 9, 10, 11, 12, 13, 14, 15, 16, 17, 18, 19, 20, 21, 22, 23, or 24 hours; or 1, 2, 3, 4, 5, 6, 7, 8, 9, 10, 11, 12, 13, or 14 days of each other.

In some aspects, the vasodilator can be any agent (e.g. compound, peptide, nucleic acid) that opens, or dilates, blood vessels. The dilation of blood vessels can allow blood to flow more easily through the blood vessels. Thus, in some aspects, the vasodilator can be administered in a therapeutically effective amount to increase the flow of blood through blood vessels in order to maintain or decrease body temperature at the target site. In some aspects, the vasodilator can be administered systemically or locally (e.g. transdermal). In some aspects, the vasodilator is nitroprusside, nitroglycerin, minoxidil, or hydralazine.

Examples of additional vasodilators that can be use include, but are not limited to, amrinone, arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, and sodium nitroprusside. Centrally acting agents include clonidine, quanaberz, and methyl dopa. Alpha adrenoceptor blocking agents include indoramin, phenoxybenzamine, phentolamine, and prazosin. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion blocking agents include pentolinium and trimetaphan. Calcium channel blockers include amlodipine, diltiazem, felodipine, isradipine, nicardipine, nifedipine, nimodipine, and verapamil. Prostaglandins including: prostacyclin, thrombuxane A2, leukotrienes, PGA, PGA1, PGA2, PGE1, PGE2, PGD, PGG, and PGH can also be used. Angiotensin II analogs can also be used and include saralasin. Other suitable vasodilators that can be used include nitroglycerin, labetalol, thrazide, isosorbide dinitrate, pentaerythritol tetranitrate, digitalis, hydralazine, diazoxide, and sodium nitroprusside, derivatives of these and combinations of the foregoing. In some aspects, the disclosed vasodilators can be a single vasodilator or the combination of two or more vasodilators.

In some aspects, the vasodilator can be administered using any of the routes of administration described throughout. In some aspects, the vasodilator can be administered intravenously or transdermally. In some aspect, contacting the target site of the subject to a vasodilator comprises transdermal administration of the vasodilator to the subject at the target site. In some aspects, contacting a target site of a subject to a vasodilator can occur by administering the vasodilator at a location other than the target site so long as the route of administration allows for vasodilation at the target site. For example, in some aspects, the vasodilator can be present in a gel, or similar form, used when applying the alternating electric field to a target site.

In some aspects, the vasodilator can be a negative pressure environment that vasodilates the vasculature. For example, using a negative pressure device to create vasodilation can be used in the extremities of a subject.

In some aspects, the increase in blood circulation is 1% greater than circulation prior to exposure to the vasodilator. In some aspect, an increase in blood circulation is 1%, 5% or 10% greater than circulation prior to exposure to the vasodilator. The increase in circulation can be measured relative to circulation prior to treatment with a vasodilator or relative to circulation after treatment with a control formulation lacking a vasodilator. In some aspect, an increase in blood circulation can be measured using laser Doppler velocimetry, which typically outputs a voltage that is proportional to the velocity of cells moving through the blood. In some aspects, an increase in circulation is any amount that maintains or decreases the temperature at a target site.

In some aspects, the target site is the subject's head, chest, or extremity.

In some aspects, the temperature at a target site after exposing the target site to an alternating electric field is not increased compared to the temperature at the target site prior to exposure to the alternating electric field. In some aspects, no increase in temperature can include a temperature that remains within less than one degree difference from the temperature prior to the alternating electric field. Thus, in some aspects, the temperature at a target site after exposing the target site to an alternating electric field is maintained. In some aspects, the temperature at the target site after exposing the target site to an alternating electric field is decreased compared to the temperature at the target site prior to exposure to the alternating electric field. In some aspects, a decrease in temperature can include a one degree drop in temperature or more.

In some aspects, the period of time, frequency or field strength of the alternating electrical field can be increased from a first exposure period of time, frequency or field strength of the alternating electrical field. For example, the vasodilators can maintain or decrease the temperature at the target site well enough to allow for an extended period of time for exposure to the alternating electrical field and/or to allow for increased frequency and/or field strength of the alternating electrical field over the exposure time period. The heat that could be caused by prolonged exposure to the alternating electrical field or exposure to the alternating electrical field at a frequency or field strength higher than normal can be dissipated by the vasodilator. Thus, in some aspects, treatment with alternating electrical fields can be altered when combined with a vasodilator.

### D. Methods of Enhancing Efficacy

Alternating electric fields have been used in a variety of treatments, such as cancer treatment. For example, alternating electric fields can be used to disrupt tumor cell mitosis. Thus, enhancing the efficacy of alternating electric fields ultimately can enhance efficacy of treatment, such as cancer treatments. The ability to control, maintain or decrease the temperature at a target site can, for example, allow the alternating electric fields to be applied for a longer duration or at a higher frequency.

Disclosed are methods of enhancing the efficacy of an alternating electric field at a target site of a subject comprising exposing a target site of the subject to an alternating electric field for a period of time, the alternating electric field having a frequency and field strength, and contacting the target site of the subject to a vasodilator, wherein the vasodilator increases circulation at the target site of the subject. In some aspects, disclosed are methods of enhancing the efficacy of an alternating electric field at a target site of a subject comprising exposing a target site of the subject to an alternating electric field for a period of time, the alternating electric field having a frequency and field strength, and administering a vasodilator to the subject, wherein the vasodilator increases circulation at the target site of the subject. Such administration can be systemic or local (e.g. transdermal).

In some aspects, enhancing the efficacy of the alternating electric field at the target site can allow for increased frequency and/or field strength, allowing for prolonged exposure period to the alternating electric field at the target site, allowing for decreased exposure period to the alternating electric field at the target site. For example, if the frequency and/or field strength can be increased, the exposure time may be able to be decreased. In some aspects, enhancing the efficacy of the alternating electric field at the target site can mean improving the end result of the alternating electric field (e.g. treating cancer or preventing mitosis of cancer cells at the target site).

In some aspects, the period of time, frequency or field strength of the alternating electrical field can be increased without increasing the temperature at the target site, thereby enhancing the efficacy of the alternating electric field at the target site.

In some aspects, the target site comprises a cancer cell. In some aspects, the cancer cell is a glioblastoma cell or lung metastatic carcinoma cell. In some aspects, the cancer cell can be any cancer cell.

In some aspects, the target site can be exposed to the alternating electric field and vasodilator simultaneously. In some aspects, the target site can be exposed to the alternating electric field and vasodilator consecutively. For example, in some aspect, the target site can be exposed to the alternating electric field prior to or after the target site is exposed to the vasodilator. In some aspects, consecutive exposure of the target site to both the alternating electric field and vasodilator can mean the exposure to each occurs within 1, 5, 10, 15, 20, 25, 30, 35, 40, 45, 50, 55, or 60 minutes; 1, 2, 3, 4, 5, 6, 7, 8, 9, 10, 11, 12, 13, 14, 15, 16, 17, 18, 19, 20, 21, 22, 23, or 24 hours; or 1, 2, 3, 4, 5, 6, 7, 8, 9, 10, 11, 12, 13, or 14 days of each other.

In some aspects, the vasodilator can be any agent (e.g. compound, peptide, nucleic acid) that opens, or dilates, blood vessels. The dilation of blood vessels can allow blood to flow more easily through the blood vessels. In some aspects, the vasodilator can be administered systemically or locally (e.g. transdermal). Thus, in some aspects, the vasodilator can be administered in a therapeutically effective amount to increase the flow of blood through blood vessels. In some aspects, the vasodilator is nitroprusside, nitroglycerin, minoxidil, or hydralazine. Examples of additional vasodilators that can be use include, but are not limited to, amrinone, arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, and sodium nitroprusside. Centrally acting agents include clonidine, quanaberz, and methyl dopa. Alpha adrenoceptor blocking agents include indoramin, phenoxybenzamine, phentolamine, and prazosin. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion blocking agents include pentolinium and trimetaphan. Calcium channel blockers include amlodipine, diltiazem, felodipine, isradipine, nicardipine, nifedipine, nimodipine, and verapamil. Prostaglandins including: prostacyclin, thrombuxane A2, leukotrienes, PGA, PGA1, PGA2, PGE1, PGE2, PGD, PGG, and PGH can also be used. Angiotensin II analogs can also be used and include saralasin. Other suitable vasodilators that can be used include nitroglycerin, labetalol, thrazide, isosorbide dinitrate, pentaerythritol tetranitrate, digitalis, hydralazine, diazoxide, and sodium nitroprusside, derivatives of these and combinations of the foregoing. In some aspects, the disclosed vasodilators can be a single vasodilator or the combination of two or more vasodilators.

In some aspects, the vasodilator can be administered using any of the routes of administration described throughout. In some aspects, the vasodilator can be administered intravenously or transdermally. In some aspect, exposing the target site of the subject to a vasodilator comprises transdermal administration of the vasodilator to the subject at the target site. In some aspects, contacting a target site of a subject to a vasodilator can occur by administering the vasodilator at a location other than the target site so long as the route of administration allows for vasodilation at the target site. For example, in some aspects, the vasodilator can be present in a gel, or similar form, used when applying the alternating electric field to a target site.

In some aspects, the vasodilator can be a negative pressure environment that vasodilates the vasculature. For example, using a negative pressure device to create vasodilation can be used in the extremities of a subject.

In some aspects, the increase in blood circulation is 1% greater than circulation prior to exposure to the vasodilator. In some aspect, an increase in blood circulation is 1%, 5% or 10% greater than circulation prior to exposure to the vasodilator. The increase in circulation can be measured relative to circulation prior to treatment with a vasodilator or relative to circulation after treatment with a control formulation lacking a vasodilator. In some aspect, an increase in blood circulation can be measured using laser Doppler velocimetry, which typically outputs a voltage that is proportional to the velocity of cells moving through the blood.

In some aspects, the target site is the subject's head, chest, or extremity.

In some aspects, the temperature at the target site after exposing the target site to an alternating electric field is not increased compared to the temperature at the target site prior to exposure to the alternating electric field. In some aspects, no increase in temperature can include a temperature that remains within less than one degree difference from the temperature prior to the alternating electric field. Thus, in some aspects, the temperature at a target site after exposing the target site to an alternating electric field is maintained. In some aspects, the temperature at the target site after exposing the target site to an alternating electric field is decreased compared to the temperature at the target site prior to exposure to the alternating electric field. In some aspects, a decrease in temperature can include a one degree drop in temperature or more.

In some aspects, the period of time, frequency or field strength of the alternating electrical field can be increased from a first exposure period of time, frequency or field strength of the alternating electrical field. For example, the vasodilators can maintain or decrease the temperature at the target site well enough to allow for an extended period of time for exposure to the alternating electrical field and/or to allow for increased frequency and/or field strength of the alternating electrical field over the exposure time period. The heat that could be caused by prolonged exposure to the alternating electrical field or exposure to the alternating electrical field at a frequency or field strength higher than normal can be dissipated by the vasodilator. Thus, in some aspects, treatment with alternating electrical fields can be altered when combined with a vasodilator.

### E. Methods of Treating

Disclosed are methods of treating cancer in a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein the target site comprises one or more cancer cells. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

Disclosed are methods of increasing apoptosis of a cancer cell in a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein the target site comprises one or more cancer cells. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

Disclosed are methods of reducing viability of cancer cells in a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein the target site comprises one or more cancer cells. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

In some aspects, the target site comprises a cancer cell. In some aspects, the cancer cell is a glioblastoma cell or lung metastatic carcinoma cell. In some aspects, the cancer cell can be any cancer cell.

In some aspects, the target site is exposed to the alternating electric field and vasodilator simultaneously. In some aspects, the target site is exposed to the alternating electric field and vasodilator consecutively. For example, in some aspect, the target site is exposed to the alternating electric field prior to or after the target site is exposed to the vasodilator. In some aspects, consecutive exposure of the target site to both the alternating electric field and vasodilator can mean the exposure to each occurs within 1, 5, 10, 15, 20, 25, 30, 35, 40, 45, 50, 55, or 60 minutes; 1, 2, 3, 4, 5, 6, 7, 8, 9, 10, 11, 12, 13, 14, 15, 16, 17, 18, 19, 20, 21, 22, 23, or 24 hours; or 1, 2, 3, 4, 5, 6, 7, 8, 9, 10, 11, 12, 13, or 14 days of each other.

In some aspects, the vasodilator can be any agent (e.g. compound, peptide, nucleic acid) that opens, or dilates, blood vessels. The dilation of blood vessels can allow blood to flow more easily through the blood vessels. Thus, in some aspects, the vasodilator can be administered in a therapeutically effective amount to increase the flow of blood through blood vessels. In some aspects, the vasodilator can be administered systemically or locally (e.g. transdermal). In some aspects, the vasodilator is nitroprusside, nitroglycerin, minoxidil, or hydralazine. Examples of additional vasodilators that can be use include, but are not limited to, amrinone, arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, and sodium nitroprusside. Centrally acting agents include clonidine, quanaberz, and methyl dopa. Alpha adrenoceptor blocking agents include indoramin, phenoxybenzamine, phentolamine, and prazosin. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion blocking agents include pentolinium and trimetaphan. Calcium channel blockers include amlodipine, diltiazem, felodipine, isradipine, nicardipine, nifedipine, nimodipine, and verapamil. Prostaglandins including: prostacyclin, thrombuxane A2, leukotrienes, PGA, PGA1, PGA2, PGE1, PGE2, PGD, PGG, and PGH can also be used. Angiotensin II analogs can also be used and include saralasin. Other suitable vasodilators that can be used include nitroglycerin, labetalol, thrazide, isosorbide dinitrate, pentaerythritol tetranitrate, digitalis, hydralazine, diazoxide, and sodium nitroprusside, derivatives of these and combinations of the foregoing. In some aspects, the disclosed vasodilators can be a single vasodilator or the combination of two or more vasodilators.

In some aspects, the vasodilator can be administered using any of the routes of administration described throughout. In some aspects, the vasodilator can be administered intravenously or transdermally. In some aspect, contacting the target site of the subject to a vasodilator comprises transdermal administration of the vasodilator to the subject at the target site. In some aspects, contacting a target site of a subject to a vasodilator can occur by administering the vasodilator at a location other than the target site so long as the route of administration allows for vasodilation at the target site. For example, in some aspects, the vasodilator can be present in a gel, or similar form, used when applying the alternating electric field to a target site.

In some aspects, the vasodilator can be a negative pressure environment that vasodilates the vasculature. For example, using a negative pressure device to create vasodilation can be used in the extremities of a subject.

In some aspects, the increase in blood circulation is 1% greater than circulation prior to exposure to the vasodilator. In some aspect, an increase in blood circulation is 1%, 5% or 10% greater than circulation prior to exposure to the vasodilator. The increase in circulation can be measured relative to circulation prior to treatment with a vasodilator or relative to circulation after treatment with a control formulation lacking a vasodilator. In some aspect, an increase in blood circulation can be measured using laser Doppler velocimetry, which typically outputs a voltage that is proportional to the velocity of cells moving through the blood.

In some aspects, the target site is the subject's head, chest, or extremity.

In some aspects, the temperature at the target site after exposing the target site to an alternating electric field is not increased compared to the temperature at the target site prior to exposure to the alternating electric field. In some aspects, no increase in temperature can include a temperature that remains within less than one degree difference from the temperature prior to the alternating electric field. Thus, in some aspects, the temperature at a target site after exposing the target site to an alternating electric field is maintained. In some aspects, the temperature at the target site after exposing the target site to an alternating electric field is decreased compared to the temperature at the target site prior to exposure to the alternating electric field. In some aspects, a decrease in temperature can include a one degree drop in temperature or more.

In some aspects, the period of time, frequency or field strength of the alternating electrical field can be increased from a first exposure period of time, frequency or field strength of the alternating electrical field. For example, the vasodilators can maintain or decrease the temperature at the target site well enough to allow for an extended period of time for exposure to the alternating electrical field and/or to allow for increased frequency and/or field strength of the alternating electrical field over the exposure time period. The heat that could be caused by prolonged exposure to the alternating electrical field or exposure to the alternating electrical field at a frequency or field strength higher than normal can be dissipated by the vasodilator. Thus, in some aspects, treatment with alternating electrical fields can be altered when combined with a vasodilator.

### F. Combination Therapies

Any of the disclosed methods can be combined with a therapeutic agent. In some aspects, a therapeutic agent can be any therapeutic that treats a condition that alternating electric fields can also treat. For example, alternating electric fields are known to treat cancer, therefore the disclosed methods can be combined with a known cancer therapeutic such as, but not limited to, chemotherapy, radiation, immunotherapy, or hormone therapy. In some aspects, a therapeutic agent can be any therapeutic that treats broad symptoms such as, but not limited to, pain medications or anti-inflammatories.

Thus, in some aspects, disclosed are methods of treating cancer in a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein the target site comprises one or more cancer cells and further administering a cancer therapeutic to the subject. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells and further administering a cancer therapeutic to the subject. In some aspects, the vasodilator and the cancer therapeutic can be administered using the same route of administration. In some aspects, the vasodilator and the cancer therapeutic can be administered using different routes of administration.

Disclosed are methods of treating cancer in a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein the target site comprises one or more cancer cells, wherein the method further comprises administering to the subject an AKT inhibitor, a mammalian target of rapamycin (mTOR) inhibitor, a Phosphatidylinositol 3-Kinase (PI3K) inhibitor, Src tyrosine kinase (Src) inhibitor, Focal adhesion kinase (Fak) inhibitor, and/or a Glycogen synthase kinase (GSK3(3) inhibitor. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

Disclosed are methods of treating cancer in a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein the target site comprises one or more cancer cells, wherein the method further comprises administering an effective amount of trastuzumab to the subject. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells, wherein the method further comprises administering to the subject an IGFR1 inhibitor, JNK inhibitor, RPS6 inhibitor or ERK inhibitor. Disclosed are methods of treating cancer in a subject comprising exposing a target site of the subject to an alternating electric field for a period of time, the alternating electric field having a frequency and field strength, and contacting the target site of the subject to a vasodilator, wherein the vasodilator increases circulation at the target site of the subject, wherein the target site comprises one or more cancer cells.

Disclosed herein are methods for increasing sensitivity of a cancer cell to alternating electric fields by administering an AKT inhibitor, a mammalian target of rapamycin (mTOR) inhibitor, a Phosphatidylinositol 3-Kinase (PI3K) inhibitor, Src tyrosine kinase (Src) inhibitor, Focal adhesion kinase (Fak) inhibitor, and/or a Glycogen synthase kinase 313 (GSK3(3) inhibitor, wherein the method further comprises administering a vasodilator to the subject. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

Disclosed are methods of increasing sensitivity of a cancer cell to alternating electric fields comprising exposing the cancer cell to an alternating electric field for a period of time, the alternating electric field having a frequency and field strength, wherein the frequency and field strength of the alternating electric field, and exposing the cancer cell to an mTOR inhibitor, AKT inhibitor, PI3K inhibitor, Src inhibitor, Fak inhibitor, and/or GSK3β inhibitor, wherein the method further comprises administering a vasodilator to the subject. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

Disclosed are methods of increasing treatment efficacy comprising applying an alternating electric field to a target site of the subject for a period of time, the alternating electric field having a frequency and field strength, wherein the target site comprises one or more cancer cells, and administering a therapeutically effective amount of one or more of an mTOR inhibitor, AKT inhibitor, PI3K inhibitor, Src inhibitor, Fak inhibitor, or GSK3β inhibitor to the subject, wherein the method further comprises administering a vasodilator to the target site. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of one or more of an mTOR inhibitor, AKT inhibitor, PI3K inhibitor, Src inhibitor, Fak inhibitor, or GSK3β inhibitor to the subject, wherein the method further comprises administering a vasodilator to the target site. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

Disclosed are methods of increasing sensitivity of a cancer cell to alternating electric fields comprising exposing the cancer cell to an alternating electric field for a period of time, the alternating electric field having a frequency and field strength, wherein the frequency and field strength of the alternating electric field, and exposing the cancer cell to an IGFR1 inhibitor, JNK inhibitor, RPS6 inhibitor or ERK inhibitor, wherein the method further comprises administering a vasodilator to the subject. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

Disclosed are methods of increasing treatment efficacy comprising applying an alternating electric field to a target site of the subject for a period of time, the alternating electric field having a frequency and field strength, wherein the target site comprises one or more cancer cells, and administering a therapeutically effective amount of one or more of an IGF1R inhibitor, JNK inhibitor, RPS6 inhibitor, and/or ERK inhibitor to the subject, wherein the method further comprises administering a vasodilator to the target site. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of one or more of an IGF1R inhibitor, JNK inhibitor, RPS6 inhibitor, and/or ERK inhibitor to the subject, wherein the method further comprises administering a vasodilator to the target site. In some aspects, disclosed are methods of treating cancer in a subject comprising exposing a target site of the subject to an alternating electric field for a period of time, the alternating electric field having a frequency and field strength, and administering a vasodilator to the subject, wherein the vasodilator increases circulation at the target site of the subject, wherein the target site comprises one or more cancer cells.

In some aspects, the vasodilator and the cancer therapeutic can be administered simultaneously. In some aspects, the vasodilator and the cancer therapeutic can be administered consecutively.

In some aspects, the vasodilator and the cancer therapeutic can be administered separately. In some aspects, the vasodilator and the cancer therapeutic can be administered together.

### G. Alternating Electric Fields

The methods disclosed herein comprise alternating electric fields. In some aspects, the alternating electric field used in the methods disclosed herein is a tumor-treating field (TTF). In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes. In some aspects, the electrodes can be applied to a subject with a layer of gel in between the electrode and the subject's skin. In some aspects, the gel can comprise a vasodilator.

In some aspects, the frequency of the alternating electric field is between 100 and 500 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 200 kHz, 300 kHz, 400 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some aspects, the field strength is at least 1 V/cm. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising equipment for applying alternating electrical fields and one or more vasodilators. The kits also can contain instructions for how to apply the alternating electrical fields.

Disclosed are kits comprising a device capable of administering an alternating electric field and optionally a vasodilator. Disclosed are kits comprising a device capable of administering an alternating electric field and optionally a vasodilator. In some aspects, the kits further comprise instructions for using the device and/or applying the alternating electrical field to a cell or a subject.

In some aspects the kits disclosed herein can further comprise instructions for using a device capable of administering an alternating electric field in combination with a vasodilator.

In some aspects, the kits disclosed herein can comprise instructions for where to apply the alternating electrical field. In some aspects, the kits disclosed herein can comprise instructions for determining a region-of-interest (ROI) within a 3D model of a portion of a subject's body, determining, based on a center of the ROI, a plane that transverses the portion of the subject's body, wherein the plane comprises a plurality of pairs of positions along a contour of the plane, adjusting, based on an anatomical restriction, one or more positions of the plurality of pairs of positions to generate a modified plane, determining, for each pair of positions of the plurality of pairs positions on the modified plane, a simulated electric field distribution, determining, based on the simulated electric field distributions, a dose metric for each pair of positions of the plurality of pairs positions, determining one or more sets of pairs of positions of the plurality of pairs of positions that satisfy an angular restriction between pairs of transducer arrays, and determining, based on the dose metrics and the one or more sets of pairs of positions that satisfy the angular restriction, one or more candidate transducer array layout maps.

In some aspects, the kits disclosed herein comprise a device capable of administering an alternating electric field, wherein the kit further comprises electrodes for applying the alternating electric field. (e.g. Optune system). In some aspects, the kits disclosed herein can further comprise instructions on where to apply the electrodes to increase the efficacy of alternating electric fields therapy. In some aspects, the kits disclosed herein further comprise instructions for conducting and analyzing measurements to determine where to apply the electrodes or where to apply the alternating electrical field.

Those skilled in the art will recognize, or be able to ascertain using no more than routine experimentation, many equivalents to the specific embodiments of the method and compositions described herein. Such equivalents are intended to be encompassed by the following claims.

### I. Embodiments

Embodiment 1. A method of increasing blood circulation at a target site of a subject comprising: (A) exposing a target site of the subject to an alternating electric field for a period of time, the alternating electric field having a frequency and field strength, and (B) contacting the target site of the subject to a vasodilator, wherein the vasodilator increases circulation at the target site of the subject.

Embodiment 2. A method of maintaining or decreasing temperature at a target site of a subject comprising: (A) exposing a target site of the subject to an alternating electric field for a period of time, the alternating electric field having a frequency and field strength, and (B) contacting the target site of the subject to a vasodilator, wherein the vasodilator increases circulation at the target site of the subject, wherein increased circulation maintains or decreases temperature at the target site.

Embodiment 3. A method of enhancing the efficacy of an alternating electric field at a target site of a subject comprising: (A) exposing a target site of the subject to an alternating electric field for a period of time, the alternating electric field having a frequency and field strength, and (B) contacting the target site of the subject to a vasodilator, wherein the vasodilator increases circulation at the target site of the subject, wherein the period of time, frequency or field strength of the alternating electrical field is increased without increasing the temperature at the target site, thereby enhancing the efficacy of the alternating electric field at the target site.

Embodiment 4. A method of treating cancer in a subject comprising: (A) exposing a target site of the subject to an alternating electric field for a period of time, the alternating electric field having a frequency and field strength, and (B) contacting the target site of the subject to a vasodilator, wherein the vasodilator increases circulation at the target site of the subject, wherein the target site comprises one or more cancer cells.

Embodiment 5. The method of any preceding embodiment, wherein the frequency of the alternating electric field is between 100 and 500 kHz.

Embodiment 6. The method of any preceding embodiment, wherein the frequency of the alternating electric field is between 180 and 220 kHz.

Embodiment 7. The method of any preceding embodiment, wherein the target site comprises a cancer cell.

Embodiment 8. The method of any preceding embodiment, wherein the cancer cell is a glioblastoma cell or lung metastatic carcinoma cell.

Embodiment 9. The method of any preceding embodiment, wherein the target site is exposed to the alternating electric field and vasodilator simultaneously.

Embodiment 10. The method of any preceding embodiment, wherein the target site is exposed to the alternating electric field and vasodilator consecutively.

Embodiment 11. The method of any preceding embodiment, wherein the vasodilator is nitroprusside, nitroglycerin, minoxidil, or hydralazine.

Embodiment 12. The method of any preceding embodiment, wherein the increase in blood circulation is 1% greater than blood circulation prior to exposure to the vasodilator.

Embodiment 13. The method of any preceding embodiment, wherein contacting the target site of the subject to a vasodilator comprises transdermal administration of the vasodilator to the subject at the target site.

Embodiment 14. The method of any preceding embodiment, wherein the target site is the subject's head, chest, or extremity.

1. A method of increasing blood circulation at a target site of a subject comprising:
a. exposing a target site of the subject to an alternating electric field for a period of time, the alternating electric field having a frequency and field strength, and
b. contacting the target site of the subject to a vasodilator, wherein the vasodilator increases circulation at the target site of the subject.

2. The method of claim 1, wherein the frequency of the alternating electric field is between 100 and 500 kHz.

3. The method of claim 1, wherein the frequency of the alternating electric field is between 180 and 220 kHz.

4. The method of claim 1, wherein the target site comprises a cancer cell.

5. The method of claim 1, wherein the cancer cell is a glioblastoma cell or lung metastatic carcinoma cell.

6. The method of claim 1, wherein the target site is exposed to the alternating electric field and vasodilator simultaneously.

7. The method of claim 1, wherein the target site is exposed to the alternating electric field and vasodilator consecutively.

8. The method of claim 1, wherein the vasodilator is nitroprusside, nitroglycerin, minoxidil, or hydralazine.

9. The method of claim 1, wherein the increase in blood circulation is 1% greater than blood circulation prior to exposure to the vasodilator.

10. The method of claim 1, wherein contacting the target site of the subject to a vasodilator comprises transdermal administration of the vasodilator to the subject at the target site.

11. The method of claim 1, wherein the target site is the subject's head, chest, or extremity.

12. A method of maintaining or decreasing temperature at a target site of a subject comprising:
a. exposing a target site of the subject to an alternating electric field for a period of time, the alternating electric field having a frequency and field strength, and
b. contacting the target site of the subject to a vasodilator,
wherein the vasodilator increases circulation at the target site of the subject, wherein increased circulation maintains or decreases temperature at the target site.

13. The method of claim 12, wherein the frequency of the alternating electric field is between 100 and 500 kHz.

14. The method of claim 12, wherein the target site comprises a cancer cell.

15.-17.

18. A method of treating cancer in a subject comprising
a. exposing a target site of the subject to an alternating electric field for a period of time, the alternating electric field having a frequency and field strength, and
b. contacting the target site of the subject to a vasodilator, wherein the vasodilator increases circulation at the target site of the subject,
wherein the target site comprises one or more cancer cells.

19. The method of claim 18, wherein the frequency of the alternating electric field is between 100 and 500 kHz.
